(19) United States
(12) Patent Application Publication
(10) Pub. No.: US 2025/0044290 A1
(43) Pub. Date: Feb. 6, 2025

(54) Biomarkers For Detecting Human Glycogen Phosphorylase Isoenzyme BB

(71) Applicant: Diagenics Group, Inc., Woburn, MA (US)

(72) Inventors: Ernest Kapetanovic, Bissen (LU); Samir Yastas, Bissen (LU)

(21) Appl. No.: 18/804,199

(22) Filed: Aug. 14, 2024

Related U.S. Application Data
(63) Continuation of application No. 16/475,876, filed on Jul. 3, 2019, now abandoned.

(30) Foreign Application Priority Data
Jan. 5, 2017 (EP) 17150385.7

(51) Int. Cl. G01N 33/573; C07K 16/40

(52) U.S. Cl. CPC G01N 33/573; C07K 16/40; G01N 2333/91097; G01N 2800/2871; G01N 2800/324; G01N 2800/368

(57) ABSTRACT

Described are an oligo-peptide sequence of any of RDHLVGRWIR (E1), IRRFKSSKFGCR (E2), RHLEIIYAINQR (E3), LIIKLVT (E4), VVGDRLKVIF (E5), any combination of E1 to E3; or combination of E4 and E5. Further described is a detecting agent for specific detecting glycogen phosphorylase iso-enzyme BB (GPBB), wherein the detecting agent is characterized by specific recognizing and binding to (1) an epitope of the GPBB comprising an oligo-peptide sequence of any of RDHLVGRWIR (E1), IRRFKSSKFGCR (E2), RHLEIIYAINQR (E3) or any combination of E1 to E3; or (2) an epitope of GPBB comprising an oligo-peptide sequence of LIIKLVT (E4), and/or VVGDRLKVIF (E5), or combination of E4 and E5.

### TECHNICAL FIELD

The present invention relates to detecting agents and methods for specifically detecting glycogen phosphorylase isoenzyme BB (GPBB) in a sample.

### BACKGROUND ART

Myocardial infarction (MI) or acute myocardial infarction (AMI), commonly known as a heart attack, occurs when blood flow stops to a part of the heart causing damage to the heart muscle. The most common symptom is chest pain or discomfort which may travel into the shoulder, arm, back, neck, or jaw. Often it is in the center or left side of the chest and lasts for more than a few minutes.

Strokes are sudden neurological disorders that occur when poor blood flow to the brain results in cell death. There are two main types of stroke: ischemic, due to lack of blood flow, and hemorrhagic, due to bleeding. Symptoms of strokes include sudden numbness or weakness of the face, arm, or leg, sudden confusion, dizziness, and sudden headache. Ischemic stroke is the combinatorial effect of many pathological processes including the loss of energy supplies, excessive intracellular calcium accumulation, oxidative stress, and inflammatory responses. The brain's ability to maintain energy demand through the pathological process involves metabolism of glycogen, which is critical for release of stored glucose.

Preeclampsia, which is a pregnancy-specific syndrome characterized by new onset hypertension and proteinuria, is a considerable obstetric problem and a significant source of maternal and neonatal morbidity and mortality.

Rapid and simple diagnosis methods of patients suffering from acute syndromes related to myocardial infarction or strokes are desired, which can provide crucially in-time information for making therapeutic decisions and adequately medical treatments. Such methods are particularly desired for circumstances, when standard medical diagnoses which require large medical instruments such as electrocardiographic device, MRI as well as CT and have to be carried out in a hospital.

Standard clinic diagnosis criteria for myocardial infarction include clinical history of ischemic type chest pain lasting for more than 20 minutes, changes in serial electrocardiography (ECG) tracings, and rise or fall of serum cardiac biomarkers such as creatine kinase-MB (CK-MB) fraction and troponin. For instance, the ST segment is used in the ECG diagnosis, which connects the QRS complex and the T wave and represents the period when the ventricles are depolarized. The ST segment is usually isoelectric, but may be depressed or elevated with myocardial infarction or ischemia. Creatine kinase-MB fraction and troponin are bio-chemical indicators for myocardial cell damage. The criteria established by the World Health Organization (WHO) defined MI as any two of the three characteristics of (a) typical symptoms (i.e., chest discomfort), (b) enzyme rise, and (c) typical ECG pattern involving the development of Q-waves (an indication of necrosed myocardium). With these criteria, which were established some years ago, the “enzyme rise” refers to the rise of serum levels of creatine kinase (CK) or its more cardiac specific isoform CK-MB. Troponin is a skeletal and smooth muscle protein useful in laboratory diagnosis of heart attack, as it is released into the bloodstream when damage to the heart muscle occurs. It is a part of the troponin complex, which binds to tropomyosin and forms a troponin-tropomyosin complex. Therefore, both CK-MB and troponin can be used as a marker for clinic diagnose of myocardial infarction and are generally considered as serum markers of necrosis.

However, the early diagnosis myocardial infarction is problematic especially in the absence of ST segment elevation, because troponin is released upon injury and is detectable in blood sample at first within 4-6 hours of coronary occlusion depending on the assay used. An accomplished Point-of-care assay as a bedside rapid test method, which is on the basis of measuring serum cardiac troponin T in patients, provides useful information to the clinician. However, as above-mentioned reasons, such an assay is not a satisfied solution in the early laboratory diagnosis of myocardial infarction despite reduction in analysis time and saving the sample transport.

Glycogen phosphorylase is a key enzyme in the regulation of glycogen metabolism and consists of three isoenzymes, namely liver isoform (GPLL), muscle isoform (GPMM), and brain/heat isoform (GPBB).

Glycogen phosphorylase (GP) is activated to degrade glycogen in response to different stimuli. Early experimental and clinical reports on GPBB kinetics following myocardial ischemic injury suggested that GPBB could be a useful diagnostic marker for early detection of acute myocardial infarction. During ischemia of heart muscle cells, an increased glycogenolysis is observed. Accordingly, the increase of glycogen phosphorylase to breakdown glycogen is observed when the heart muscle is damaged. In this case, the GPBB is soluble in the cytoplasm and released into the bloodstream. The release of GPBB into the blood within the first few hours after myocardial ischemia is a direct indication of lacking oxygen supply to the heart muscle. Clinical studies have shown a higher sensitivity of GPBB within the first four hours after symptom of myocardial ischemia in comparison with other cardiac markers.

Ischemia of brain tissue results in activation of membrane-bound GPBB into active and soluble form. The soluble isoform of GPBB initiates glycogen breakdown. Thus, GPBB is also an interesting marker for early diagnosis of strokes.

Higher maternal plasma GPBB concentration in preeclampsia patients has been reported, which are very likely to be associated with uteroplacental hypoxia leading to the conversion of GPBB into soluble form and to the release of placental GPBB into the bloodstream. Accordingly, such finding strongly suggests that maternal plasma GPBB can be a useful marker in the detection of preeclampsia.

Moreover, in contrast to the markers of necrosis such as CK-MB and troponin, GPBB is an ischemic marker, which is considered to improve early diagnosis in acute coronary syndrome. During the process of ischemia, GPBB is converted into a soluble form and is released into the blood. A rapid rise in blood levels can be seen in myocardial infarction due to a lack of oxygen in the heart. GPBB is elevated from 1 to 3 hours after the process of ischemia. Thus, GPBB has been emerging as marker for early diagnosis of myocardial infarction.

For example, WO 2008/064903 A teaches the use of antibodies against the epitope sequences GRWIRTQQHYYERDPKRIYYLSLEFYMGRTLQNTM, IFNQKIVNGWQVEEADDWLRYGNPWEKARP or GLGDVAEVRKSFNRHLHFT-LVKDRNVATPRDYFFA or DSMATLGLAAYGYGIRYEFG of GPBB.

In addition, WO 2012/171878 A discloses a method for ascertaining the ischemic level of patients with suspected stroke, which comprises a step of determining the concentration of GPBB in a blood sample.

Moreover, US 2016/0116472 A relates to assays and methods of diagnosing strokes using GPBB as the biomarker. Specifically, assays and methods are described herein for distinguishing an ischemic brain condition from myocardial infarction.

DE 102013106254 A discloses a method for diagnosing preeclampsia or evaluating risk of preeclampsia in pregnant women. The method is characterized by obtaining a blood sample of the pregnant subject and determining a concentration and/or activity of GPBB in the sample.

However, these prior art do not provide fully useful detecting agents, which can precisely determine the level of GPBB in patient samples.

### SUMMARY OF THE INVENTION

### Problem to be Solved by the Invention

In a first aspect, the problem to be solved by the present invention is to define the part of the amino acid sequence of GPBB which can be used in its quantitative detection in patient samples.

In a second aspect, the problem to be solved by the present invention is to provide detecting agents for quantitative detecting the level of GPBB in patient samples, which enable an early diagnosis of myocardial infarction and strokes.

### Solution of the Underlying Object

The first aspect of the present invention is solved by the following oligo-peptide sequences of the enzyme GPBB: RDHLVGRWIR (E1), IRRFKSSKFGCR (E2), RHLEIIYAINQR (E3), LIIKLVT (E4), VVGDRLKVIF (E5), any combination of E1 to E3; or combination of E4 and E5.

The second aspect of the present invention is solved by detecting agents for the quantitative detecting of the level of GPBB which recognize and bind specifically to certain epitopes of GPBB which are identified by means of an epitope mapping. The epitope sequences according to the present invention are
1) an epitope of the GPBB comprising an oligo-peptide sequence of any of RDHLVGRWIR (E1), IRRFKSSKFGCR (E2), RHLEIIYAINQR (E3) or any combination of E1 to E3;
or
2) an epitope of GPBB comprising an oligo-peptide sequence of LIIKLVT (E4), and/or VVG-DRLKVIF (E5), or combination of E4 and E5.

### Advantageous Effect of the Invention

The advantage of the present invention is that detecting agents are provided that can quantitatively determine the level of GPBB in a sample. Therefore, an early diagnosis of diseases which relates to GPBB level in a sample of a patient is possible.

### Means for Solving the Problem

### Oligo-Peptide Sequence

By using the below specified epitope mapping, the above-mentioned oligo-peptide sequence is identified which is suitable for detecting GPBB as biomarker in blood by the following detecting agents.

### Detecting Agents

A detecting agent for specific detecting glycogen phosphorylase isoenzyme BB (GPBB), wherein the detecting agent is characterized by specific recognizing and binding to
1) an epitope of the GPBB comprising an oligo-peptide sequence of any of RDHLVGRWIR (E1), IRRFKSSKFGCR (E2), RHLEIIYAINQR (E3) or any combination of E1 to E3;
or
2) an epitope of GPBB comprising an oligo-peptide sequence of LIIKLVT (E4), and/or VVGDRLKVIF (E5), or combination of E4 and E5.

In another aspect of the present invention, the detecting agent for specific detecting GPBB can be any kind of molecules including a protein, a polypeptide, and a polynucleotide which specifically bind to the epitope of the GPBB. The detecting agents according to the present invention can be used as a first binding agent.

In a further aspect of the present invention, the detecting agent for specific detecting GPBB is preferably an antibody or polypeptide, which binds specifically to one of the above-mentioned epitope sequences alone or two or more of the mentioned epitope sequences.

In a preferable aspect of the present invention, the detecting agent does not bind to glycogen phosphorylase isoenzyme LL or glycogen phosphorylase isoenzyme MM.

In another aspect of the present invention, the detecting agent is used for in vitro detecting GPBB by means of an immunoassay.

In another aspect of the present invention, the use of the detecting agent enables a quantitative determination of the GPBB level by means of the immunoassay.

According to another aspect of the present invention, the detecting agent is used for detecting GPBB level in a blood, serum or plasma sample.

According to a further aspect of the present invention, the detecting agent is preferably used for determine GPBB level in the blood sample.

In one aspect of the present invention, the detecting agent is used for diagnosis of myocardial infarction.

In another aspect of the present invention, the detecting agent is used for diagnosis of stroke.

In a further aspect of the present invention, the detecting agent is used for diagnosis of preeclampsia.

In a preferable aspect of the present invention, the detecting agent is a mouse monoclonal antibody 326 G5.

According to one aspect of the present invention, the detecting agent specifically binds at least to one epitope selected from the group of the oligo-peptide sequences of RDHLVGRWIR (E1), IRRFKSSKFGCR (E2), RHLEIIYAINQR (E3) or any combination of E1 to E3.

According to another aspect of the present invention, the detecting agent specifically binds to the epitope comprising a combination of the oligo-peptide sequence E1 and E2 (E1+E2).

According to a further aspect of the present invention, the detecting agent specifically binds to the epitope comprising a combination of the oligo-peptide sequence E1 and E3 (E1+E3).

According to a further aspect of the present invention, the detecting agent specifically binds to the epitope comprising a combination of the oligo-peptide sequence E2 and E3 (E2+E3).

According to one aspect of the present invention, the detecting agent specifically binds to the epitope comprising a combination of the oligo-peptide sequence E1, E2 and E3 (E1+E2+E3).

In another preferable aspect of the present invention, the detecting agent is a mouse monoclonal antibody 329 B6.

According to one aspect of the present invention, the specifically binds to the epitope comprising the oligo-peptide sequence of LIIKLVT (E4), and/or VVGDRLKVIF (E5), or combination of E4 and E5.

According to another aspect of the present invention, the detecting agent specifically binds to the epitope comprising combination of the oligo-peptide sequence E4 and E5 (E4+E5).

In a further preferable aspect of the present invention, the detecting agent can be used in combination with any other detecting agent, which does not bind to the said epitope sequence.

In another preferable aspect of the present invention, another biomarker CK-MB and/or troponin.

### Method for Specific Detecting GPBB

In one aspect of the present invention, the method for specific detecting GPBB is characterized by the following steps:
(a) providing a detecting agent specifically recognizing and binding to
1) an epitope of the GPBB comprising an oligo-peptide sequence of any of RDHLVGRWIR (E1), IRRFKSSKFGCR (E2), RHLEIIYAINQR (E3) or any combination of E1 to E3;
or
2) an epitope of GPBB comprising an oligo-peptide sequence of LIIKLVT (E4), and/or VVGDRLKVIF (E5), or combination of E4 and E5,
(b) bringing the detecting agent in contact with a sample.

The sample according to the present invention is preferably blood sample, serum sample, or plasma sample.

In another aspect of the present invention, the method is an immunoassay method, which is further characterized by comprising the following steps:
(a) providing the detecting agent as a first binding agent which can specifically bind to the above-mentioned epitope sequence of GPBB,
(b) bringing the detecting agent in contact with the sample,
(c) providing a second binding agent that specifically binds to the first binding agent.

The first binding agent described herein is the detecting agent for specific detecting the above-mentioned GPBB, which includes any molecule, i.e., antibodies or antibody fragments, peptides or peptide fragments, enzymes, proteins, peptide complexes, peptide and carbohydrate complexes, nucleic acid molecules, or other chemical entities, as long as it has a binding specificity.

In another aspect of the present invention, the said method is able to quantitatively determine a GPBB level in the sample.

In a further aspect of the present invention, the method includes a further step of comparing the determined GPBB level with a reference GPBB level.

In one aspect of the present invention, the method is used to for analyzing blood sample, serum sample, or plasma sample.

In a preferable aspect of the present invention, the first binding agent is preferably an antibody which binds specifically to the epitope of the GPBB.

In one aspect of the present invention, the method according to the present invention comprises a second binding agent. The second binding agent can be conjugated or unconjugated with a detection probe, which includes any molecule, i.e., antibodies or antibody fragments, peptides or peptide fragments, enzymes, proteins, peptide complexes, peptide and carbohydrate complexes, nucleic acid molecules, or other chemical entities, as long as it has a binding specificity to the first binding agent.

In one embodiment, the second binding agent is preferably a non-conjugated secondary antibody which can specifically bind to the first binding agent (detecting agent). The detection of immunoassay is carried out by adding of a detection probe. A detection probe, which can bind to the second binding agent, is selected form detectable enzymes, prosthetic groups, paramagnetic groups, fluorescent materials, luminescent materials, bioluminescent materials, radioactive materials, disperse dyes, gold particles, or a combination thereof.

In another aspect of the present invention, a conjugated secondary antibody, this can specifically bind to the first binding agent (detecting agent).

is preferably used as the second binding agent. Any kind of above-mentioned detection probe is herein conjugated on the secondary antibody.

In one aspect of the present invention, the sample is collected from a person who has symptoms of myocardial infarction.

In a preferable aspect of the present invention, the sample is collected from a person who has symptoms of myocardial infarction who has symptoms of stroke.

In a further aspect of the present invention, the sample is collected from a person who has symptoms of preeclampsia.

In one aspect of the present invention, a kit of parts can be used for determining a GPBB level in a sample, which comprises the detecting agent according to the present invention.

In another aspect of the present invention, a kit of parts comprising the detecting agent is used for diagnosis of myocardial infarction.

In a further aspect of the present invention, a kit of parts comprising the detecting agent is used for diagnosis of stroke.

In a further aspect of the present invention, a kit of parts comprising the detecting agent is used for diagnosis of preeclampsia.

### BRIEF DESCRIPTION OF THE DRAWINGS

FIG. 1 shows the results of a control scan after 15 minutes pre-swelling in washing buffer and 30 minutes incubation in blocking buffer.

FIG. 2 shows detection of epitope sequences of GPBB, onto which antibody 326 G5 binds.

To detect epitope sequences of GPBB, on which antibody 326 G5 binds, the microarrays were incubated with mouse monoclonal antibody 326 G5 at concentrations of 100 μg/mL, 500 μg/mL (scans not shown) and 1000 μg/mL. Staining with secondary and control antibodies was followed. Afterward, microarray was read out at scanning intensities of 7/7 (red/green); for a better data overview, the baselines of the intensity plot were leveled.

FIG. 3 (FIG. 3) shows detection of epitope sequences of GPBB, on which antibody 329 B6 binds.

### DETAILED DESCRIPTION OF THE INVENTION

The detecting agent described herein is first binding agent for specific detecting GPBB, which includes any molecule, i.e., antibodies or antibody fragments, peptides or peptide fragments, enzymes, proteins, peptide complexes, peptide and carbohydrate complexes, nucleic acid molecules, or other chemical entities, so long as it has a binding specificity that binds to.

The first binding agents include preferable antibodies or antibody fragments, including monoclonal, polyclonal, humanized, human, chimeric, recombinant, bispecific, multispecific antibodies, or a combination thereof. The antibody fragments may comprise Fab, Fab (2)′ Fc, Fv, single chain antibody, or a combination thereof.

According to the present method, the second binding agents herein includes any molecule, i.e., antibodies or antibody fragments, peptides or peptide fragments, enzymes, proteins, peptide complexes, peptide and carbohydrate complexes, nucleic acid molecules, or other chemical entities, as long as it has a binding specificity that binds to, or interacts with the first binding agent. The second binding agent is preferably as antibody and can be either conjugated or non-conjugated not-conjugated with a detection probe described below.

A detection probe is used in the immunoassay, which can bind to the second binding agent. The detection probe is selected from form detectable enzymes, prosthetic groups, paramagnetic groups, fluorescent materials, luminescent materials, bioluminescent materials, radioactive materials, disperse dyes, gold particles, or a combination thereof.

The immunoassay used according to the present invention covers any of a biochemical test that measures the presence or concentration of a macromolecule or a small molecule in a solution through the use of an antibody or an antigen. Such assay methods are for instance ELISA including Sandwich ELISA (catch and detect), MELISA, CEDIA, immunoscreening, lateral flow test, magnetic immunoassay, radioimmunoassay, surround optical fiber immunoassay (SOFIA), but are not limited to these.

According to the present invention, one or more further detecting agents, which do not bind to the said epitope sequence, can be applied in combination with the detecting agent having the claimed epitopes. Such preferred detecting agents include CK-MB and/or troponin.

According to the present invention, a kit by using the detecting agents or method according the present invention can be used for determining a GPBB level in a sample.

Moreover, the results of the epitope mapping provide another aspect of the present invention in order to synthesize further detecting agents.

Based on the knowledge of the three-dimensional structure of GPBB and the sequence of GPBB, it is possible to use the above-mentioned epitopes as a cassette information (E1, E2, E3, etc. location in the gene/protein) to know where in the protein respective high affinity binding sites (habs) are for both “detecting agents” (e.g. for the mouse monoclonal Antibodies 326 G5 and 329 B6).

This method for preparing further detecting agents is characterized by the following process steps:
(I) step (I): preparing a peptide, comprising at least one oligo-peptide sequence selected from the group consisting of RDHLVGRWIR (E1), IRRFKSSKFGCR (E2), RHLEIIYAINQR (E3, LIIKLVT (E4) and VVG-DRLKVIF (E5);
(II) step (II): peptide immunization based on the peptide resulting from step (I);
(III) step (III): identification of suitable detecting agents resulting from step (II) against the peptide of step (I) and/or the enzyme GPBB.

### Step (I):

Based on the new knowledge of the epitopes RDHLVGRWIR (E1), IRRFKSSKFGCR (E2), RHLEIIYAINQR (E3), LIIKLVT (E4) and VVGDRLKVIF (E5), it is possible to prepare different peptides which can be used as starting point in the preparation of further detecting agents according to the present invention. These peptides might either:
a. a sequence of E1-E2-E3-E4-E5 (i.e a full cassette for the mouse monoclonal Antibodies 326 G5 and 329 B6) with the natural interim amino acid and/or gene sequence (the gene/protein starting at E1 ending at E5);
b. a sequence of E1-E2-E3-E4-E5 (i.e a full cassette for the mouse monoclonal Antibodies 326 G5 and 329 B6) with any sequence spacer between the different epitopes (the sequence spacers are different as compared with the natural interim amino acid and/or gene sequence (embodiment a. above));
c. a sequence of E1-E2-E3-E4-E5 (i.e a full cassette for the mouse monoclonal Antibodies 326 G5 and 329 B6; RDHLVGRWIRIRRFKSSKFGCRRHLEIIYAINQRLIIKLVTVVGDRLKVIF) without any sequence spacer between the different epitopes);
d. a sequence of a part of E1-E2-E3-E4-E5 (i.e a shorter cassette for the mouse monoclonal Antibodies 326 G5 and 329 B6) with the natural interim amino acid and/or gene sequence (e.g.
d.1 a sequence of two epitopes selected from the group consisting of E1-E2, E2-E3, E3-E4, and E4-E5;
d.2a sequence of three epitopes selected from the group consisting of E1-E2-E3, E2-E3-E4, and E3-E4-E5;
d.3a sequence of four epitopes selected from the groups consisting of E1-E2-E3-E4 and E2-E3-E4-E5;
with the natural interim amino acid and/or gene sequence between the respective epitopes);
e. a sequence of a part of E1-E2-E3-E4-E5 (i.e a shorter cassette for the mouse monoclonal Antibodies 326 G5 and 329 B6) with any sequence spacer between the different epitopes (the sequence spacers are different as compared with the natural interim amino acid and/or gene sequence (embodiment d. above)) (e.g.
e.1 a sequence of two epitopes selected from the group consisting of E1-E2, E2-E3, E3-E4, and E4-E5;
e.2a sequence of three epitopes selected from the group consisting of E1-E2-E3, E2-E3-E4, and E3-E4-E5;
e.3a sequence of four epitopes selected from the groups consisting of E1-E2-E3-E4 and E2-E3-E4-E5;
with any sequence spacer between the different epitopes (the sequence spacers are different as compared with the natural interim amino acid and/or gene sequence (embodiment d. above)));
f. a sequence of a part of E1-E2-E3-E4-E5 without any sequence spacer in between (e.g.
f.1 a sequence of two epitopes selected from the group consisting of E1-E2, E2-E3, E3-E4, and E4-E5;
f.2 a sequence of three epitopes selected from the group consisting of E1-E2-E3, E2-E3-E4, and E3-E4-E5;
f.3 a sequence of four epitopes selected from the groups consisting of E1-E2-E3-E4 and E2-E3-E4-E5;
without any sequence spacer in between.

In case the peptides comprise non-natural spacers between the different epitopes, these spacers can be selected based on protein design conclusions.

The above-mentioned peptides can be prepared by using classical organic chemistry in which multiple amino acids are linked via amide bonds (for example by liquid-phase synthesis or solid-phase synthesis) or by protein biosynthesis respectively recombinant synthesis.

### Step (II):

Based on the peptide resulting from step (I), peptide immunization is carried out.

The resulting mG (mini-GPBB) of Step (I) are used as an antibody inducing antigen (AIA) which will generate antibody clones.

Step (II) can be carried out in an eukaryotic cell (such as a yeast) or prokaryotic (in a bacteria). One further possibility is to carry out the peptide immunization in a human cell.

### Step (III):

The antigens prepared by the immunization of step (II) are then identified as suitable detecting agents against one of the peptides of step (I) and/or the enzyme GPBB.

The antigens are selected based on the best high affinity and high yield non cross resistant antibody clones.

In the present invention, GPBB can be detected in a human (human GPBB) or in an animal (animal GPBB).

### EXAMPLES

### Example 1: Preparation of Antibodies which Specifically Bind to GPBB

Antibodies binding to GPBB are known. However, such known bindings are usually non-specific and the known antibodies also bind to isoenzymes MM and LL, which have similar amino acid sequences. Thus, it is desired that the detecting agents according to the present invention only specifically bind to the isoform BB to avoid any cross-reactions with isoenzymes MM and LL, and thus, the binding specificity of produced antibodies is increased.

In order to produce such specific antibodies, Balb-c mice were immunized with purified enzyme. The spleen cells of these mice were fused with mouse myeloma cells line Sp 2/0 after the appearance of serum antibodies. The resulting Hybridomas were cultured. The antibody-producing hybridomas were re-cloned and tested for their accurate usability for the intended use. The specific hybridoma clones were expanded and stored in liquid nitrogen. The production of antibodies is via standard techniques. The resulting antibodies were tested for their specific binding against to the GPBB.

### Example 2: Reactivity Test (Monoclonal Antibody Against Solid-Phase-Immobilized GPBB)

The reactivity and specificity of the produced monoclonal antibodies (mAb), which are supposed specifically binding to GPBB are tested by means of enzyme linked immunosorbent assay (ELISA).

The microtiter plates were coated with soluble GPBB and GPMM with a protein concentration of 3 μg/mL. The enzymes were diluted with a buffer of phosphate-buffered saline (pH 7.4, 0.15 M NaCl). The coating was performed at 4° C. After 24 hours of wetting term, the plates were treated with standard blocking agent to avoid non-specific binding. The blocking is carried by sealing the plates in aluminum pocket containing drying agent and incubating at 4° C. overnight. Afterward, the plates are directly used for testing the selected monoclonal antibodies by means of ELISA. The ELISA tests are carried out as following procedures:
1) supernatants of cell culture (1C4G1(A1), 1H12D9) and the purified mAb ((A11H3 (A4), 1E8G9 (B2) and 4A5F3 (13C3)) were adjusted to having the same albumen or IgG content and titrated in a dilution plate of 2 μg/mL to 0.03 μg/mL;
2) 200 μg/mL of these solution form dilution plate were transferred by using multichannel pipette into the microtiter plates coated with GPBB. In order to avoid any effects due to different incubation time, each microtiter plates coated with GPBB were stuck together, so that parallel processing was possible;
3) the microtiter plates containing mixed solution was incubated for 60 minutes at 37° C.;
4) the microtiter plates were washes five times to remove unbound components;
5) anti-mouse IgG-Peroxidase antibody (1 μg/mL, 200 μL per well) was added and incubated for another 60 minutes at 37° C.;
6) the microtiter plates were washes five times to remove unbound secondary antibody;
7) measurement was carried out by adding 200 μL of peroxidase substrate tetramethylbenzidine (TMB) liquid substrate with SeramunBlau® to each well and reading of released signal.

### Results

In the ELISA test, none of produced monoclonal antibody shows reactivity against immobilized GPMM. Except for antibody mAb 2A11H3 (A4), the reactivity in respect of extinction of these antibodies against to solid-phase bound GPBB was comparable to those results obtained earlier. The parallel test of mAb 329 B6 and 326 G5 showed reproducible extinction yield of insoluble GPBB compared to those results obtained previously.

### Example 3: Reactivity Test (Monoclonal Antibody Against Membrane-Immobilized GPBB)

The reactivity of the produced mAb, which are supposed specifically binding to GPBB are further tested by means of dot blot (binding of analyte on a nitrocellulose membrane and detection with antibody).

In order to exclude the unrecognizable cross-reactivity with GPMM, which is possibly due to the different conformation depending on the carrier material in the ELISA, mAb with abbreviated designation A1, A4, B2, 13C3, and D9 was selected for further characterization of their reactivity against GPBB and GPMM on dot blot. For this purpose, 1.1 μL samples of GPBB and GPMM, which contain about 1 μg protein, was dropped on a nitrocellulose membrane and dried overnight. Subsequently, the nitrocellulose membranes containing GP samples were blocked for 2 hours with blocking agent and dried overnight.

mAb 329 B6 and 326 G5 were tested parallel in the same manner.

For the dot blot, the monoclonal antibodies were tested according to the following procedures:
1) the content of protein and IgG was adjusted and titrated of concentration in 1000 ng/mL to 7.8 ng/mL;
2) 1 mL dilution of IgG protein from each dilution level was added into blot tank, which includes a corresponding blot membrane containing GP;
3) the blot membranes in blot tank were incubated for 60 minutes at room temperature on a shaker;
4) the blot membranes were washed 3 times with washing buffer and each time for 5 minutes;
5) anti-mouse IgG-HRP (1 μg/mL, 1 mL per each dot membrane) was added; the dot membrane was further incubated for 60 minutes at room temperature on a shaker;
6) the blot membrane was washed 3 times with washing buffer and each time for 5 minutes;
7) measuring was carried out by using SeramunBlau® and visually evaluated.

### Result

All seven monoclonal antibodies react with nitrocellulose-bound GPBB. On the other hand, none of the seven selected monoclonal antibodies reacts with the membrane-bound GPMM.

### Example 4: Identification of Epitope of GPBB by Epitope Mapping

Epitopes (binding site) of antibodies on their target antigens can be identified by experimental procedures, which is called epitope mapping.

For instance, array-based oligo-peptide scanning can be used for identifying epitopes. Such a method is based on combinatorial chemistry, in which procedures for mapping and characterizing epitopes involving the synthesis of overlapping peptides and analysis of the peptides in enzyme-linked immunosorbent assays (ELISAs).

### Preparing Microarray

To identify the epitope of GPBB, on which the mouse monoclonal Antibodies 326 G5 and 329 B6 bind, the sequence of GPBB (UniProt ID: P11216, see enclosed protein sequence according to WIPO standard st.25) was elongated by neutral GSGSGSG linkers at the C- and N-terminus to avoid truncated peptides. The elongated antigen sequence was translated into 7, 10 and 13 amino acid peptides with peptide-peptide overlaps of 6, 9 and 12 amino acids. After peptide synthesis, all peptides were cyclized via a thioether linkage between a C-terminal cysteine side chain thiol group and an appropriately modified N-terminus. The resulting GPBB peptide microarrays contained 2,544 different cyclic constrained peptides printed in duplicate (5,088 peptide spots) and were framed by additional HA (YPYDVPDYAG, 146 spots) and c-myc (EQKLISEEDL 146 spots) control peptides.

Buffer and detection conditions for immuno-detection are provided as follows:
Samples: Mouse monoclonal antibodies 326 G5 (2 mg/mL) and 329 B6 (2.4 mg/mL)
Washing Buffer: PBS, pH 7.4 with 0.005% Tween 20 (2×10 second after each assay) Blocking Buffer: Rockland blocking buffer MB-070 (30 minutes before the first assay) Incubation Buffer: Washing buffer with 10% blocking buffer
Assay Conditions: Antibody concentrations of 10 μg/mL, 100 μg/mL, 500 μg/mL and 1000
μg/mL in incubation buffer; incubation for 16 hours at 4° C. and shaking at 140 rpm
Secondary Antibody: Goat anti-mouse IgG (H+L) DyLight 680 (1:5000); 45 minutes staining in incubation buffer at room temperature
Control Antibody: Mouse monoclonal anti-HA (12CA5) DyLight 800 (1:2000); 45 min staining in incubation buffer at room temperature
Scanner: LI-COR Odyssey Imaging System; scanning offset 0.65 mm, resolution 21 μm, scanning intensities of 7/7 (red=700 nm/green=800 nm)

### Immuno-Detection of Microarrays

Pre-staining of one of the human GPBB peptide microarrays was done with the secondary antibody goat anti-mouse IgG (H+L) DyLight 680 (1:5000 dilution) in incubation buffer to investigate background interactions with the antigen-derived cyclic constrained peptides that could interfere with the main assays. Subsequent incubation of other human GPBB peptide microarrays with the mouse monoclonal antibody samples at concentrations of 10 μg/mL (data not shown) 100 μg/mL, 500 μg/mL and 1000 μg/mL in incubation buffer was followed by staining with the secondary antibody and read-out at scanning intensities of 7/7 (red/green). To avoid any interference with the secondary antibody goat anti-mouse IgG (H+L) DyLight 680, the control staining of the HA epitopes with control antibody mouse monoclonal anti-HA (12CA5) DyLight 800 was done afterwards as internal quality control to confirm the assay quality and the peptide microarray integrity.

Quantification of spot intensities and peptide annotation were based on the 16-bit grayscale tiff files at scanning intensities of 7/7 that exhibit a higher dynamic range than the 24-bit colorize tiff files. Microarray image analysis was done with PepSlide® Analyzer and summarized in the Excel files listed in Material and Methods. A software algorithm breaks down fluorescence intensities of each spot into raw, foreground and background signal and calculates average median foreground intensities and spot-to-spot deviations of spot duplicates. Based on averaged median foreground intensities, an intensity map was generated and interactions in the peptide map highlighted by an intensity color code with red for high and white for low spot intensities. A maximum spot-to-spot deviation of 40% is tolerated, otherwise the corresponding intensity value was zeroed.

Averaged spot intensities of the assays with the antibody samples against the antigen sequence from the N- to the C-terminus of human GPBB to visualize overall spot intensities and signal-to-noise ratios were further plotted. The intensity plots were correlated with pep-tide and intensity maps as well as with visual inspection of the microarray scans to identify the epitopes of the mouse antibody samples. In case it was not clear if a certain amino acid contributed to antibody binding, the corresponding letters were written in gray.

### Control Scan

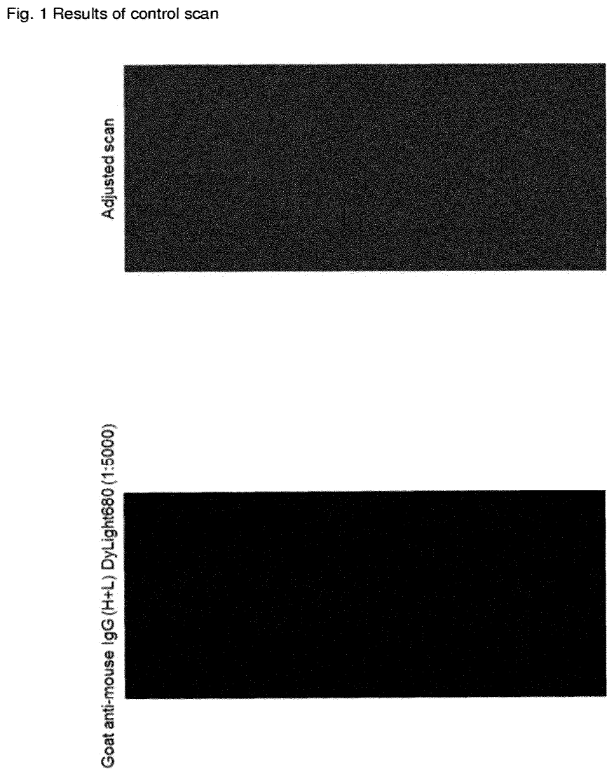

The results of a control scan are represented in FIG. 1. After 15 minutes pre-swelling in washing buffer and 30 minutes incubation in blocking buffer, one of the GPBB peptide microarrays was initially incubated with the secondary goat anti-mouse IgG (H+L) DyLight 680 antibody (1:5000 dilution) for 45 minutes at room temperature to analyze background interactions with the antigen-derived cyclic constrained peptides.

At scanning intensities of 7/7 (red/green), there is not any no any background interaction of the secondary antibody with the GPBB peptides to be observed, even upon significant increase of brightness and contrast (see adjusted scan). Data quantification with PepSlide® Analyzer was hence omitted.

Detecting Epitope Sequences of GPBB, on which Antibody 326 G5 Binds

To detect epitope sequences of GPBB, on which antibody 326 G5 binds, the microarrays were incubated with mouse monoclonal antibody 326 G5 at concentrations of 100 μg/mL, 500 μg/mL (scans not shown) and 1000 μg/mL. Staining with secondary and control antibodies was followed. Afterward, microarray was read out at scanning intensities of 7/7 (red/green); for a better data overview, the baselines of the intensity plot were leveled.

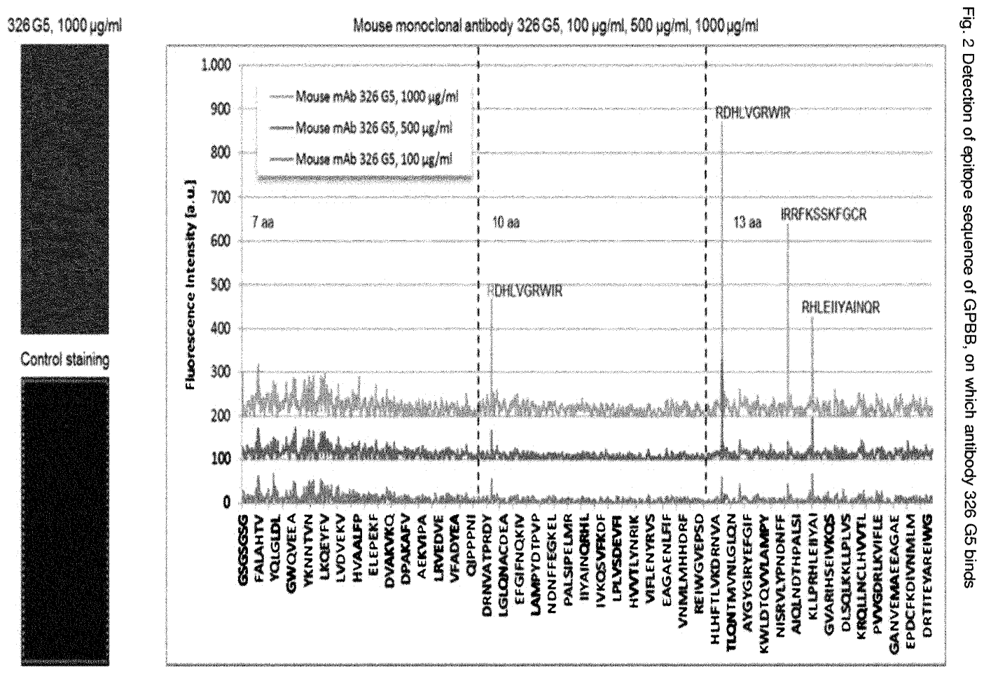

The results are schematic represented in FIG. 2 which show clear response against peptides with the consensus motif RDHLVGRWIR at the highest antibody concentrations; the interactions with peptides with the consensus motifs IRRFKSSKFGCR and RHLEIIYAINQR were attributed to cross-reactions, which indicates low signal-to-noise ratios and well-defined staining of the frame of HA control peptides.

Detecting Epitope Sequences of GPBB, on which Antibody 329 B6 Binds

To detect epitope sequences of GPBB, on which antibody 329 B6 binds, microarrays were incubated with mouse monoclonal antibody 329 B6 at concentrations of 100 μg/mL, 500 μg/mL (scans not shown) and 1000 μg/mL. Staining with secondary and control antibodies was followed. Afterward, microarray was read out at scanning intensities of 7/7 (red/green); for a better data overview, the baselines of the intensity plot were leveled.

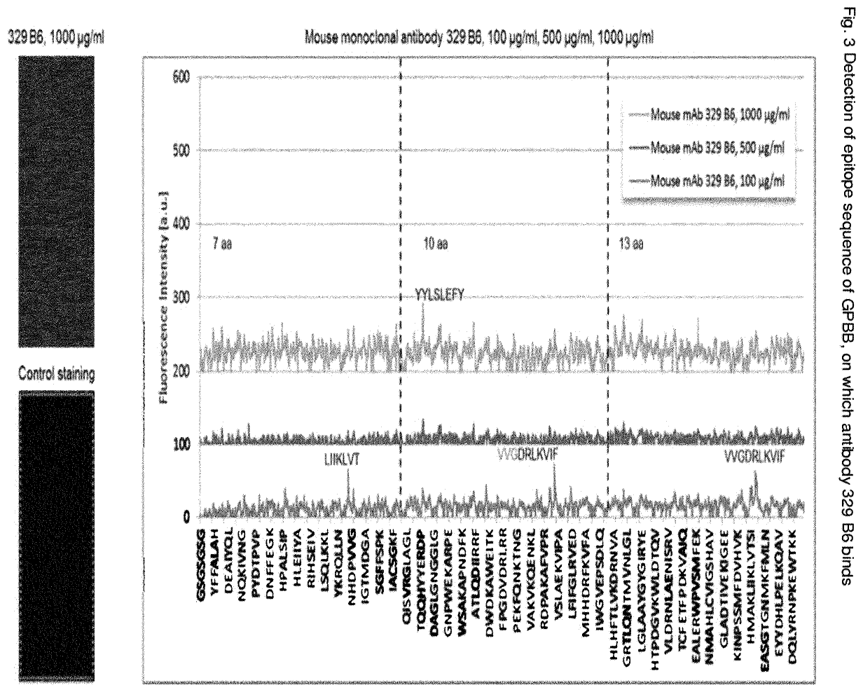

The results are schematic represented in FIG. 3, which show response against peptides with the consensus motif VVGDRLKVIF at a concentration of 100 μg/mL; the interaction with peptides with the consensus motifs LIIKLVT was attributed to a cross-reaction, which indicated low signal-to-noise ratios; well-defined staining of the frame of HA control peptides

The PEPperMAP® Conformational Epitope Mappings of mouse antibodies 326 G5 and 329 B6 were performed with 7, 10 and 13 amino acid cyclic constrained peptides of human GPBB with peptide-peptide overlaps of 6, 9 and 12 amino acid. The corresponding human GPBB peptide microarrays were incubated with the antibody samples at concentrations of 10 μg/mL, 100 μg/mL, 500 μg/mL and 1000 μg/mL in incubation buffer followed by staining with secondary and control antibodies as well as read-out with a LI-COR Odyssey Imaging System. Quantification of spot intensities and peptide annotation were done with PepSlide® Analyzer.

Pre-staining of one of the human GPBB peptide microarrays with the secondary goat anti-mouse IgG (H+L) DyLight 680 (1:5000 dilution) did not show any background interaction with the cyclic GPBB peptides. In contrast, incubation with the mouse antibody samples resulted in the following observations. observations:

Mouse mouse monoclonal antibody 326 G5 exhibited response just against the 10 amino acid and 13 amino acids of GPBB peptides with the consensus motif RDHLVGRWIR; other interactions were found for the 13 amino acid of cyclic constrained peptides with the consensus motifs IRFKSSKFGCR and RHLEIIYAINQR, presumably due to a cross-reaction based on a sequence similarities; since the proposed epitope had a length of 9 or 10 amino acids, we were not able to identify any response with the 7 amino acids of GPBB peptides peptides.

Mouse mouse monoclonal antibodies 329 B6 showed response with very low signal-to-noise ratios and just slightly above the assay background against the 10 amino acids and 13 amino acids of GPBB peptides with the consensus motif VVGDRLKVIF; since the proposed epitope had a length of 8 or 10 amino acids, we were not able to identify any response with the 7 amino acids of GPBB peptides except for a background interaction with the hydrophobic peptide LIIKLVT; it should be pointed out, however, that the response against peptides with the consensus motif VVGDRLKVIF was only observed at a concentration of 100 μg/mL, but not at higher concentrations of 100 μg/mL and 500 μg/mL, in consideration with the low signal-to-noise ratios.

1-42. (canceled)

43: A method for preparing detecting agents, characterized by the following process steps:
(I) step (I):
preparing a peptide, comprising at least one oligo-peptide sequence selected from the group consisting of RDHLVGRWIR (E1), IRRFKSSKFGCR (E2), RHLEIIYAINQR (E3), LIIKLVT (E4) and VVG-DRLKVIF (E5);
(II) step (II):
peptide immunization based on the peptide resulting from step (I);
(III) step (III):
identifying suitable detecting agents resulting from step (II) against the peptide of step (I) and/or the enzyme GPBB.

44: The method according to claim 43, wherein the peptides are selected from the group consisting of:
(a) a sequence of E1-E2-E3-E4-E5 having a natural interim amino acid and/or gene sequence; or
(b) a sequence of E1-E2-E3-E4-E5 having any sequence spacer between the different epitopes; or
(c) a sequence of E1-E2-E3-E4-E5 lacking any sequence spacer between the different epitopes; or
(d) a sequence of a part of E1-E2-E3-E4-E5 having a natural interim amino acid and/or gene sequence, selected from the group consisting of
(d.1) a sequence of two epitopes selected from the group consisting of (i) E1-E2, (ii) E2-E3, (iii) E3-E4, and (iv) E4-E5; or
(d.2) a sequence of three epitopes selected from the group consisting of (i) E1-E2-E3, (ii) E2-E3-E4, and (iii) E3-E4-E5; or
(d.3) a sequence of four epitopes selected from the group consisting of (i) E1-E2-E3-E4 and (ii) E2-E3-E4-E5;
having a natural interim amino acid and/or gene sequence between the respective epitopes; or
(e) a sequence of a part of E1-E2-E3-E4-E5 having any sequence spacer between the different epitopes, selected from the group consisting of
(e.1) a sequence of two epitopes selected from the group consisting of (i) E1-E2, (ii) E2-E3, (iii) E3-E4, and (iv) E4-E5; or
(e.2) a sequence of three epitopes selected from the group consisting of (i) E1-E2-E3, (ii) E2-E3-E4, and (iii) E3-E4-E5; or
(e.3) a sequence of four epitopes selected from the groups consisting of (i) E1-E2-E3-E4 and (ii) E2-E3-E4-E5;
having any sequence spacer between the different epitopes; or
(f) a sequence of a part of E1-E2-E3-E4-E5 without any sequence spacer in between, selected from the group consisting of
(f.1) a sequence of two epitopes selected from the group consisting of (i) E1-E2, (ii) E2-E3, (iii) E3-E4, and (iv) E4-E5; or
(f.2) a sequence of three epitopes selected from the group consisting of (i) E1-E2-E3, (ii) E2-E3-E4, and (iii) E3-E4-E5; or
(f.3) a sequence of four epitopes selected from the groups consisting of (i) E1-E2-E3-E4 and (ii) E2-E3-E4-E5;
lacking any sequence spacer in between.

45: The method according to claim 43, characterized in that the peptide immunization in step (II) is carried out in a cell selected from the group consisting of (i) eukaryotic cell, (ii) yeast cell, (iii) prokaryotic cell, (iv) bacterial cell, and (v) human cell.

46: The method according to claim 44, characterized in that the peptide immunization in step (II) is carried out in a cell selected from the group consisting of (i) eukaryotic cell, (ii) yeast cell, (iii) prokaryotic cell, (iv) bacterial cell, and (v) human cell.
